(19) United States
(12) Patent Application Publication
(10) Pub. No.: US 2016/0008449 A1
(43) Pub. Date: Jan. 14, 2016

(54) CROSS-PROTECTION OF BOVINES AGAINST B. trehalosi INFECTION BY A MULTI-VALENT VACCINE

(71) Applicant: ZOETIS SERVICES LLC, Florham Park, NJ (US)

(72) Inventors: Terry Lynn Bowersock, Lawton, MI (US); Ryan N. Dunn, Hampton, NJ (US); Randy Dean Leyh, Richland, MI (US); Klaas Okkinga, Chatham, NJ (US)

(21) Appl. No.: 14/775,741

(22) PCT Filed: Mar. 13, 2014
(86) PCT No.: PCT/US2014/025655

§ 371 Date: Sep. 14, 2015

Related U.S. Application Data
(60) Provisional application No. 61/791,571, filed on Mar. 15, 2013.

(51) Int. Cl. A61K 39/02; A61K 39/155; A61K 39/12; A61K 39/265; C12N 7/00

(52) U.S. Cl. CPC A61K 39/0208; A61K 39/265; C12N 7/00; A61K 39/12; A61K 39/155; C12N 2710/16034; C12N 2770/24334; C12N 2760/18634; C12N 2760/18534; A61K 2039/70

(57) ABSTRACT

The invention provides a method of protecting a bovine susceptible to Bibersteinia trehalosi, the method comprising administering to said bovine an effective amount of a vaccine comprising a Mannheimia haemolytica antigen, and further comprising one or more of Infectious Bovine Rhinotracheitis Virus, Bovine Viral Diarrhea Virus, Parainfluenza-3 Virus, and Bovine Respiratory Syncytial Virus.

### FIELD OF THE INVENTION

The present invention relates to combination vaccines and methods for treating or preventing diseases or disorders in an animal caused by infection by Bibersteinia trehalosi (B. trehalosi).

### BACKGROUND

Bovine respiratory disease (BRD) complex is the most significant health problem of the beef industry. In 1991, an estimated loss of $624 million occurred, due to costs of treatment, production loss, and death. BRD complex is a multifactorial infection, having many contributing pathogens, both viral and bacterial. The infectious agents implicated in BRD include, without limitations, Bovine Viral Diarrhea Virus (BVDV) Types 1 and 2, Infectious Bovine Rhinotracheitis Virus (IBRV), Bovine Respiratory Syncytial Virus (BRSV), Parainfluenza Virus (PI-3), and Mannheimia haemolytica. It has relatively recently been discovered that infection by B. trehalosi can result in symptoms of Bovine Respiratory disease. Most current anti-BRD vaccines on the market do not include antigens against B. trehalosi. Accordingly, there is a need in the art for compositions and methods to protect bovines against BRD caused by B. trehalosi.

### SUMMARY OF THE INVENTION

The inventors have surprisingly discovered that a bovine (e.g., a cow, bull, steer, heifer, or calf) may be protected from B. trehalosi infection by administering a multivalent vaccine comprising a Mannheimia haemolytica bacterin-toxoid, and further comprising one or more antigens, including Infectious Bovine Rhinotracheitis Virus, Bovine Viral Diarrhea Virus, Parainfluenza-3 Virus, or Bovine Respiratory Syncytial Virus. In certain embodiments, in addition to the M. haemolytica antigen such as, for example, an attenuated live bacteria, a toxoid or a bacterin-toxoid, the vaccine may comprise Infectious Bovine Rhinotracheitis Virus, Bovine Viral Diarrhea Virus, Parainfluenza-3 Virus, and Bovine Respiratory Syncytial Virus.

In different embodiments of the invention, the viral component of the vaccine may comprise killed and/or live-attenuated viruses.

In certain embodiments, the vaccine used in the methods of the invention comprises a killed Mannheimia haemolytica antigen.

In certain embodiments, the vaccine further comprises an adjuvant. In some embodiments, the adjuvant may comprise a saponin, a sterol, a quaternary ammonium and a polyacrylic polymer. In other embodiments, the adjuvant may be a combination of O/W emulsion (e.g., AMPHIGEN®) and aluminum.

### DETAILED DESCRIPTION OF SELECTED EMBODIMENTS

For a better explanation of the invention, the following non-limiting definitions are provided.

The term “adjuvant”, as used herein, means a pharmacological or immunological agent that modifies the effect of other agents, such as a drug or immunogenic composition. Adjuvants are often included in immunogenic compositions or vaccines to enhance the recipient's immune response to a supplied antigen. See below for a further description of adjuvants.

“Antigen”, as used herein, means killed, attenuated or inactivated bacteria, viruses, fungi, parasites or other microbes. The term “antigen” also refers to a molecule that contains one or more epitopes (linear, conformational or both), that upon exposure to a subject, will induce an immune response that is specific for that antigen. An epitope is the specific site of the antigen which binds to a T-cell receptor or specific B-cell antibody, and typically comprises about 3 to about 20 amino acid residues. The term “antigen” can also refer to subunit antigens-antigens separate and discrete from a whole organism with which the antigen is associated in nature. The term “antigen” also refers to antibodies, such as anti-idiotype antibodies or fragments thereof, and to synthetic peptide mimotopes that can mimic an antigen or antigenic determinant (epitope). The term “antigen” also refers to an oligonucleotide or polynucleotide that expresses an antigen or antigenic determinant in vivo, such as in DNA immunization applications.

The term “bacterin”, as used herein, means a suspension of bacteria which has been killed or inactivated. Said inactivation can occur via various methods, including chemical, heat, ultraviolent, and other means. The term “toxoid”, as used herein, refers to an inactivated toxin. Inactivation can be accomplished either by chemical, e.g. formalin, or heat treatment. The term “bacterin-toxoid”, as used herein, refers to preparation consisting of an inactivated or killed bacteria (bacterin), combined with an inactivated toxin produced by that bacteria (toxoid).

The term “bovine”, as used herein, means a diverse group of medium- to large-sized ungulates, generally having cloven hoofs, and at least one of the sexes having true horns. Bovines include, but are not limited to, domestic cattle, bison, African buffalo, water buffalo, yak, and four-horned or spiral-horned antelope.

The term “immunogenic composition”, as used herein, means a composition that generates an immune response (i.e., has immunogenic activity) when administered alone, or with a veterinarily-acceptable carrier, to an animal. The immune response can be a cellular immune response mediated primarily by cytotoxic T-cells, or a humoral immune response mediated primarily by helper T-cells, which in turn activate B-cells, leading to antibody production.

The terms “pathogen” or “pathogenic microorganism”, as used herein, mean a microorganism—for example a virus, bacterium, fungus, protozoan, or helminth—which is capable of inducing or causing a disease, illness, or abnormal state in an animal.

The terms “prevent”, “preventing” or “prevention”, and the like, as used herein, mean to inhibit the replication of a microorganism, to inhibit transmission of a microorganism, or to inhibit a microorganism from establishing itself in its host. These terms, and the like, can also mean to inhibit or block one or more signs or symptoms of infection.

The terms “therapeutic” or “treatment”, as used herein, encompass the full spectrum of treatments for a disease or disorder. By way of example, a “therapeutic” agent of the invention may act in a manner, or a treatment may result in an effect, that is prophylactic or preventive, including those that incorporate procedures designed to target animals that can be identified as being at risk (pharmacogenetics), or in a manner that is ameliorative or curative in nature, or may act to slow the rate or extent of the progression of at least one symptom of a disease or disorder being treated.

The term “therapeutically effective amount” (or “effective amount”), as used herein, means an amount of an active ingredient, e.g., an agent used in the methods of the present invention, sufficient to effect beneficial or desired results when administered to a subject or patient. An effective amount can be administered in one or more administrations, applications or dosages. A therapeutically effective amount of a composition may be readily determined by one of ordinary skill in the art.

The term “veterinarily acceptable carrier”, as used herein, refers to a carrier medium that does not interfere with the effectiveness of the biological activity of the active ingredient, and is not toxic to the veterinary subject to whom it is administered.

As noted above, the inventors have surprisingly discovered that a complex multivalent vaccine comprising a Mannheimia haemolytica antigen, such as a toxoid, a bacterin-toxoid, or a modified live antigen, and further comprising one or more viral antigens, including IBRV, BVDV, PI-3, and BRSV, is effective for treating or preventing diseases or disorders in an animal caused by infection by B. trehalosi.

Advantageously, products containing the aforementioned ingredients are commercially available. For example, a product comprising the M. haemolytica bacterin-toxoid recited above would be One Shot® (Zoetis Inc.; New Jersey). A product comprising the viral antigens recited above would be Bovi-Shield GOLD 5 (Zoetis Inc.). In other embodiments, the vaccine may also include additional antigens of bacterial and/or viral origin. Such additional antigens include, without limitations, Campylobacter fetus, Leptospira canicola, Leptospira grippotyphosa, Leptospira borgpetersenii hardjo-prajitno, Leptospira icterohaemmorrhagiae, Leptospira borgpetersenii hardjo-bovis, Leptospira bratislava, and Leptospira interrogans pomona.

The use of the antigens recited above is well-documented, but specific strains which are particularly advantageous for use in vaccines of the instantly claimed methods are described below.

In certain embodiments, the vaccine compositions used in the methods of the present invention include an effective amount of one or more of the above-described BVDV viruses, preferably cpBVDV-2 strain 53637 (ATCC No. PTA-4859); cpBVDV-1 strain 5960 (cpBVDV-1 strain 5960-National Animal Disease Center, United States Department of Agriculture; Ames, Iowa); cpBVDV-1 strain NADL (PLEASE PROVIDE ACCESSION NUMBER IF KNOWN), IBRV strain C-13 and PI3 designated as EBK-1/EBHt-1, IBRV ts mutant strain RBL 106 (National Institute of Veterinary Research; Brussels, Belgium); PI-3 ts mutant strain RBL 103 (RIT; Rixensart, Belgium); BRSV strain 375 (Veterinary Medical Research Institute; Ames, Iowa). Purified viruses can be used directly in a vaccine composition, or preferably, the viruses can be further attenuated by way of chemical inactivation or serial passaging in vitro. In certain preferred embodiments, the viruses in vaccines used in the methods of the instant invention are modified-live viruses. In other embodiments, the viruses are killed. Combinations of live-attenuated and killed viruses are also possible.

In certain embodiments, the vaccine used in the instant invention contains cpBVDV-1 strain NADL, cpBVDV-2 strain 53637 IBRV strain C-13 and PI3 designated as EBK-1/EBHt-1.

The antigens used in the methods of the instant invention can be attenuated or inactivated prior to inclusion in a vaccine. Methods of attenuation and inactivation are well known to those skilled in the art. Methods for attenuation include, but are not limited to, serial passage in cell culture, ultraviolet irradiation, and chemical mutagenesis. Methods for inactivation include, but are not limited to, treatment with formalin, betapropriolactone (BPL) binary ethyleneimine (BEI), sterilizing radiation, or other methods known to those skilled in the art. Inactivation by formalin can be performed by mixing the suspension containing the microorganism with 37% formaldehyde, to a final formaldehyde concentration of 0.05%. The mixture is stirred constantly for approximately 24 hours at room temperature. The mixture containing the inactivated microorganism is then tested to confirm complete inactivation.

Inactivation of viruses by BEI can be performed by mixing the virus suspension with 0.1 M BEA (2-bromo-ethylamine in 0.175 N NaOH), to a final BEA concentration of 1 mM. The virus-BEA mixture is stirred constantly for approximately 48 hours at room temperature, followed by the addition of 1.0 M sodium thiosulfate to a final concentration of 0.1 mM. (Binary ethyleneimine-BEI— the primary inactivating agent, is generated in situ when the BEA is neutralized by the addition of sodium thiosulfate.) Mixing is continued for an additional two hours. The inactivated viral mixture is tested for residual live virus by assaying for growth on a suitable cell line.

For inactivation of bacteria by BEA, BEA is added directly to the production culture to a final concentration of no less than 4 mM. The culture is maintained with agitation at 37° C.±2° C. for 12-24 hours. The inactivated bacterial mixture can then be tested for residual live bacteria to confirm complete inactivation. In other embodiments, the antigen may be inactivated using formalin, at a concentration of about 0.1% for 6-7 days at 2-7° C.

The bacterial component(s) of the instant vaccine can also be inactivated by other methods well known in the art and described elsewhere. In some embodiments, inactivated bacterins may further be heat treated to inactivate lipase activity while still retaining an acceptable antigenic activity. See, e.g., US Patent Publication 20100285057. Without limitations, the bacterins useful in the vaccines useful in the methods of the present invention may be formed by culturing the bacterium of interest, and then killing the bacteria to produce a bacterin containing a variety of components, including immunogenically active agents, such as, for example, cell wall components.

Typically, an immunogenic composition or vaccine contains between about 1×10² and about 1×10¹² viral or bacterial particles, or between about 1×10³ and about 1×10¹¹ particles, or between about 1×10⁴ and about 1×10¹⁰ particles, or between about 1×10⁵ and about 1×10⁹ particles, or between about 1×10⁶ and about 1×10⁸ particles. The precise amount of a microorganism in an immunogenic composition or vaccine effective to provide a protective effect can be determined by a skilled artisan. The vaccines used in the methods of the instant invention may advantageously be formulated with veterinarily-acceptable carriers, including all solvents, dispersion media, coatings, adjuvants, stabilizing agents, diluents, preservatives, antibacterial and antifungal agents, isotonic agents, adsorption delaying agents, immunomodulators, and the like.

Diluents can include water, saline, dextrose, ethanol, glycerol, and the like. Isotonic agents can include sodium chloride, dextrose, mannitol, sorbitol, and lactose, among others. Stabilizers include albumin, among others. Preservatives include merthiolate, among others, known to the skilled artisan.

Suitable adjuvants used to enhance an immune response include, without limitation, MPL™ (3-O-deacylated monophosphoryl lipid A; Corixa; Hamilton, Mont.), which is described in U.S. Pat. No. 4,912,094, hereby incorporated by reference. Also suitable for use as adjuvants are synthetic lipid A analogs or aminoalkyl glucosamine phosphate compounds (AGP), or derivatives or analogs thereof, which are available from Corixa (Hamilton, Mont.), and which are described in U.S. Pat. No. 6,113,918, which is hereby incorporated by reference. One such AGP is 2-[(R)-3-Tetradecanoyloxytetradecanoylamino]ethyl 2-Deoxy-4-O-phosphono-3-O—[(R)-3-tetradecanoyoxytetradecanoyl]-2-[(R)-3-tetradecanoyloxytetradecanoyl-amino]-b-D-glucopyranoside, which is also known as 529 (formerly known as RC529). This adjuvant is formulated as an aqueous form or as a stable emulsion.

Still other adjuvants include mineral oil and water emulsions, aluminum salts (alum), such as aluminum hydroxide, aluminum phosphate, etc., Amphigen, Avridine, L121/squalene, D-lactide-polylactide/glycoside, pluronic polyols, muramyl dipeptide, killed Bordetella, saponins, such as Stimulon™ QS-21 (Antigenics; Framingham, Mass.), described in U.S. Pat. No. 5,057,540, which is hereby incorporated by reference, and particles generated therefrom such as ISCOMS (immunostimulating complexes), Mycobacterium tuberculosis, bacterial lipopolysaccharides, synthetic polynucleotides such as oligonucleotides containing a CpG motif (U.S. Pat. No. 6,207,646, which is hereby incorporated by reference), a pertussis toxin (PT), or an E. coli heat-labile toxin (LT), particularly LT-K63, LT-R72, PT-K9/G129; see, e.g., International Patent Publication Nos. WO 93/13302 and WO 92/19265, incorporated herein by reference.

Also useful as adjuvants are cholera toxins and mutants thereof, including those described in published International Patent Application number WO 00/18434 (wherein the glutamic acid at amino acid position 29 is replaced by another amino acid other than aspartic acid, preferably a histidine). Similar CT toxins or mutants are described in published International Patent Application number WO 02/098368 (wherein the isoleucine at amino acid position 16 is replaced by another amino acid, either alone or in combination with the replacement of the serine at amino acid position 68 by another amino acid; and/or wherein the valine at amino acid position 72 is replaced by another amino acid). Other CT toxins are described in published International Patent Application number WO 02/098369 (wherein the arginine at amino acid position 25 is replaced by another amino acid; and/or an amino acid is inserted at amino acid position 49; and/or two amino acids are inserted at amino acid positions 35 and 36).

A number of cytokines or lymphokines have been shown to have immune-modulating activity, and thus may be used as adjuvants. These include, but are not limited to, the interleukins 1-α, 1-β, 2, 4, 5, 6, 7, 8, 10, 12 (see, e.g., U.S. Pat. No. 5,723,127), 13, 14, 15, 16, 17 and 18 (and its mutant forms), the interferons-α, β and γ, granulocyte-macrophage colony stimulating factor (see, e.g., U.S. Pat. No. 5,078,996 and ATCC Accession Number 39900), macrophage colony stimulating factor, granulocyte colony stimulating factor, GSF, and the tumor necrosis factors α and β. Still other adjuvants useful in this invention can include a chemokine, including without limitation, MCP-1, MIP-1α, MIP-1β, and RANTES. Adhesion molecules, such as a selectin, e.g., L-selectin, P-selectin, and E-selectin, may also be useful as adjuvants. Still other useful adjuvants include, without limitation, a mucin-like molecule, e.g., CD34, GlyCAM-1 and MadCAM-1, a member of the integrin family such as LFA-1, VLA-1, Mac-1 and p 150.95, a member of the immunoglobulin superfamily such as PECAM, ICAMs, e.g., ICAM-1, ICAM-2 and ICAM-3, CD2 and LFA-3, co-stimulatory molecules such as CD40 and CD40L, growth factors including vascular growth factor, nerve growth factor, fibroblast growth factor, epidermal growth factor, B7.2, PDGF, BL-1, and vascular endothelial growth factor, receptor molecules including Fas, TNF receptor, Flt, Apo-1, p 55, WSL-1, DR3, TRAMP, Apo-3, AIR, LARD, NGRF, DR4, DR5, KILLER, TRAIL-R2, TRICK2, and DR6. Still another adjuvant molecule includes Caspase (ICE). See, also International Patent Publication Nos. WO98/17799 and WO99/43839, incorporated herein by reference.

While the amounts and concentrations of adjuvants and additives useful in the context of the present invention can readily be determined by the skilled artisan, the present invention contemplates the use of compositions comprising from about 50 mg to about 2000 mg of adjuvant, and preferably about 500 mg per 2 ml dose of the vaccine composition. In another preferred embodiment, the present invention contemplates the use of vaccine compositions comprising from about 1 mg/ml to about 60 mg/ml of antibiotic, and more preferably less than about 30 mg/ml of antibiotic.

Immunization protocols can be optimized using procedures well known in the art. A single dose can be administered to animals, or, alternatively, two or more inoculations can take place with intervals of two to ten weeks. Depending on the age of the animal, the immunogenic or vaccine composition can be re-administered. For example, the present invention contemplates the vaccination of healthy cattle prior to six months of age and revaccination at six months of age. In other embodiments, the combination vaccine is desirably administered twice to the animal; once at about 1 to about 3 months of age, and once at about 1 to 4 weeks later. The present invention also contemplates semiannual revaccinations with a single dose and possibly, a revaccination prior to breeding. In another set of embodiments, particularly adapted to female bovines, the first administration is performed about 5 weeks prior to breeding. The second administration is performed about 2 weeks prior to breeding. Administration of subsequent vaccine doses is preferably done on an annual basis. Animals vaccinated before the age of about 6 months could be revaccinated after 6 months of age. Administration of subsequent vaccine doses is preferably done on an annual basis.

In accordance with the present invention, administration can be achieved by known routes, including the oral, intranasal, topical, transdermal, and parenteral (e.g., intravenous, intraperitoneal, intradermal, subcutaneous or intramuscular). The typical route of administration will be intramuscular or subcutaneous injection of between about 0.1 and about 5 ml of vaccine. The vaccine compositions used in the methods of the present invention can also include additional active ingredients e.g., those described in WO 9512682, WO 9955366, U.S. Pat. No. 6,060,457, U.S. Pat. No. 6,015,795, U.S. Pat. No. 6,001,613, and U.S. Pat. No. 5,593,873.

The vaccines used in the methods of the present invention can be prepared for administration in the form of, for example, liquids, powders, aerosols, tablets, capsules, enteric-coated tablets or capsules, or suppositories. The formulation of the vaccine ultimately depends on the route of administration chosen by the practitioner. Thus, the vaccines may also include, but are not limited to, suspensions, solutions, emulsions in oily or aqueous vehicles, pastes, and implantable sustained-release or biodegradable formulations.

In one embodiment of a formulation for injections, the active ingredient is provided in dry (i.e., powder or granular) form for reconstitution with a suitable vehicle (e.g., sterile pyrogen-free water) prior to parenteral administration of the reconstituted composition. Other useful formulations include those which comprise the active ingredient in microcrystalline form, in a liposomal preparation, or as a component of a biodegradable polymer system. Compositions for sustained release or implantation may comprise pharmaceutically acceptable polymeric or hydrophobic materials, such as an emulsion, an ion exchange resin, a sparingly soluble polymer, or a sparingly soluble salt. Modified release formulations include delayed, sustained, pulsed, controlled, targeted and programmed release. Thus compounds used in the methods of the present invention can be formulated as a solid, semi-solid, or thixotropic liquid for administration as an implanted depot, providing modified release of the active compound. Examples of such formulations include drug-coated stents and poly(dl-lactic-coglycolic)acid (PLGA) microspheres.

Vaccines used in the methods of the present invention can also be administered topically to the skin or mucosa—that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions; liposomes can also be used. Typical carriers include alcohol, water, mineral oil, liquid petrolatum, white petrolatum, glycerin, polyethylene glycol and propylene glycol. Penetration enhancers can be incorporated; see, for example, Finnin and Morgan, J. Pharm. Sci, 88 (10):955-958 (1999). Other means of topical administration include delivery by electroporation, iontophoresis, phonophoresis, sonophoresis and microneedle or needle-free (e.g. Powderject™, Bioject™, etc.) injection. Formulations for topical administration can be designed to be immediate and/or modified release. Modified release formulations include delayed, sustained, pulsed, controlled, targeted and programmed release.

Vaccines can also be administered intranasally or by inhalation, typically in the form of a dry powder (either alone or as a mixture, for example, in a dry blend with lactose, or as a mixed component particle, for example, mixed with phospholipids, such as phosphatidylcholine), from a dry powder inhaler, or as an aerosol spray from a pressurized container, pump, spray, atomizer (preferably an atomizer using electrohydrodynamics to produce a fine mist). It can also be administered via a nebulizer, with or without the use of a suitable propellant, such as 1,1,1,2-tetrafluoroethane or 1,1,1,2,3,3,3-heptafluoropropane. For intranasal use, the powder can comprise a bioadhesive agent, for example, chitosan or cyclodextrin. The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is generally micronized to a size suitable for delivery by inhalation (typically less than about 5 microns). This can be achieved by any appropriate comminuting method, such as spiral jet milling, fluid bed jet milling, supercritical fluid processing (to form nanoparticles), high pressure homogenization, or spray drying.

Capsules (made, for example, from gelatin or hydroxypropylmethylcellulose), blisters, and cartridges for use in an inhaler or insufflators, can be formulated to contain a powder mix of the compound of the vaccines used in the methods of the present invention. A suitable powder base could be lactose or starch, and a performance modifier could be 1-leucine, mannitol, or magnesium stearate. The lactose can be anhydrous, or in the form of the monohydrate. Other suitable excipients include dextran, glucose, maltose, sorbitol, xylitol, fructose, sucrose and trehalose.

A suitable solution formulation for use in an atomizer, using electrohydrodynamics to produce a fine mist, can contain from about 1 μg to about 20 mg of the compound of the invention per actuation, and the actuation volume can vary from about 1 μl to about 100 μl. In another embodiment, the amount of compound per actuation can range from about 100 μg to about 15 mg, or from about 500 μg to about 10 mg, or from about 1 mg to about 10 mg, or from about 2.5 μg to about 5 mg. In another embodiment, the actuation volume can range from about 5 μl to about 75 μl, or from about 10 μl to about 50 μl, or from about 15 μl to about 25 μl. A typical formulation can comprise the compound of the invention, propylene glycol, sterile water, ethanol and sodium chloride. Alternative solvents which can be used instead of propylene glycol include glycerol and polyethylene glycol.

The invention will now be described with reference to the following non-limiting example. The example is illustrative only, and is not intended to limit the remainder of the disclosure in any way.

### Example 1

### Efficacy of a Multivalent Bovine Vaccine Against a Bibersteinia Trehalosi Challenge

The objective of the study was to demonstrate efficacy, against a virulent Bibersteinia trehalosi respiratory challenge in calves, of a vaccine containing the following modified live viruses: Infectious Bovine Rhinotracheitis Virus, Bovine Viral Diarrhea Virus, Parainfluenza-3 Virus, and Bovine Respiratory Syncytial Virus; as well as a Mannheimia haemolytica Toxoid (IBR—BVD-PI3-BRSV-MH).

Seventy-four Holstein calves were enrolled into one of three treatment groups. All animals were clinically healthy, and not previously vaccinated against M. haemolytica or P. multocida. Prior to inclusion in the study, animals were screened for serum antibodies to both M. haemolytica leukotoxin and P. multocida outer membrane proteins. Animals were seronegative for M. haemolytica leukotoxin antibodies (LKT antibody titers <1:3200), and P. multocida outer membrane proteins (OMP titers <1:2400). In addition, animals were not persistently infected with Bovine Viral Diarrhea Virus (BVDV-PI), as determined by laboratory analysis on ear notches.

TABLE 1

| Treat- | Investigational | | Vaccination | | | |
|---|---|---|---|---|---|---|
| ment | Veterinary | | | Dose/ | Challenge | |
| Group | Product¹ | N | Day | Route | Day | Dose² |
| T01 | IBR-BVD-PI3-BRSV | 37 | 0 | 2 | 21 | 120 mL; |
| T02 | IBR-BVD-PI3- | 37 | | mL/SQ³ | | 60 mL |
| | BRSV-MH | | | | | flush |
| NTX | N/A | 3 | N/A | N/A | N/A | N/A |

¹IBR-BVD-PI3-BRSV = Bovine Rhinotracheitis-Virus Diarrhea-Parainfluenza3-Respiratory Syncytial Virus Vaccine, Modified Live Virus; IBR-BVD-PI3-BRSV-MH = Bovine Rhinotracheitis-Virus Diarrhea-Parainfluenza3-Respiratory Syncytial Virus Vaccine, Modified Live Virus-Mannheimia Haemolytica Bacterin-Toxoid
²Trans-tracheal dosing
³SQ = subcutaneously

On days −1 through 19, all animals were observed for general health. On day 0, animals received 2 mL of the appropriate vaccine (Table 1) by subcutaneous administration midway between the ear and the point of the shoulder in the right neck. The NTX animals were not vaccinated.

Animals were observed for undesirable systemic reaction associated with vaccination (depression, trembling, and/or tachypnea) within four hours after vaccination by the Investigator. On days 20 through 27, all animals were observed for clinical signs of respiratory disease, and rectal temperatures were collected. Moribund animals were euthanized after a blood sample was collected.

On day 21, calves were challenged with 120 mL of a virulent B. trehalosi culture, followed by a 60 mL flush of media culture. Challenge administration was conducted in the study rooms. Prior to challenge administration, the hair over the trachea was clipped from each animal, and the trachea was disinfected using alcohol wipes. The animals remained standing for challenge. Two mL of lidocaine was infused subcutaneously over a 1-2 cm diameter area over the trachea. A 16 gauge trochar was directed caudally through the skin, and into the trachea at the spot of local anesthesia. The cannula was inserted through the trochar to the bifurcation of the trachea, and was then retracted 2-4 cm to ensure that it was in the lower trachea and not a major bronchus. The challenge material was then infused through the cannula, followed by a 60 mL flush of media. The cannula was then removed.

Blood samples were collected from each animal on study days −1 (pre-vaccination), 20 and 27 (or day of euthanasia/necropsy). Samples were allowed to clot at room temperature until processed to serum, divided into two aliquots, and then submitted for testing or stored frozen. Serum samples were assayed for antibodies to M. haemolytica leukotoxin.

Nasal swabs were collected from animals prior to vaccination and challenge (days −1 and 20) to be cultured for bacterial growth. Lung swabs and lung tissue was collected from animals on day of necropsy. Lung swabs were cultured for B. trehalosi, P. multocida, M. haemolytica, H. somni, and A. pyogenes. Lung tissue was collected and frozen.

On the day of euthanasia (day 27), the animals were humanely euthanized, and lung lesion scores were determined. The lungs were removed and scored for percent pneumonic lesions, swabbed, and a tissue sample collected. Animals that died or were euthanized post-challenge but prior to last day of challenge were necropsied and scored as described above.

The study was considered valid if B. trehalosi is isolated from the lungs at least 40% of the control animals. Efficacy was considered demonstrated if the lower 95% confidence interval of the stratified mitigated fraction calculated for lung lesion score was >0 in T02, and the median lung lesion score was ≧10% in T01. If insufficient lung lesion scores (LLSs) occur in the control group, the efficacy of the Mannheimia haemolytica Bacterin-Toxoid fraction was demonstrated post-challenge if the lower bound of the 95% confidence interval of the prevented fraction from calculated for mortality was >0 for treatment group T02 compared to group T01. Mortality was summarized by treatment. Frequency distributions for whether an animal died were calculated for each treatment. Mortality was summarized with the prevented fraction with 95% confidence limits.

### Results.

The animals were healthy throughout the study. The general health observations showed that no animals were recorded as being abnormal during the vaccination phase of the study. Within 4 hours after vaccination, no animals were observed with undesirable systemic reactions.

The study was valid, because 26 of the 37 (70.3%) control animals were positive for B. trehalosi in lung cultures.

The B. trehalosi challenge caused mortality in 35.1% of the control animals (T01), and 13.5% of the group T02 calves (Table 2). A vaccine effect in group T02 calves was observed, with preventive fraction for mortality of 0.615 (95% CI: 5.5, 89.8).

TABLE 2

| | | | Prevented | 95% |
|---|---|---|---|---|
| | | | Fraction | Confidence |
| Treatment | Mortality | Percent | (T01 vs T02) | Interval |
| T01 | 13/37 | 35.1 | 0.615 | 5.5, 89.8 |
| IBR-BVD-PI3-BRSV | | | | |
| T02 | 5/37 | 13.5 | | |
| IBR-BVD-PI3-BRSV- | | | | |
| MH | | | | |

The median percent of lung lesions in group T01 was 47.35%, compared to 32.35% in treatment group T02. (Table 3) The mitigated fraction estimates for the lung lesions was 0.379 (95% CI: 32.1, 41.7), indicating a positive effect from vaccination.

TABLE 3

| | % Lung | | Mitigated | 95% |
|---|---|---|---|---|
| | Lesion | | Fraction | Confidence |
| Treatment | (median) | Range | (T01 vs T02) | Interval |
| T01 | 47.35 | 7-72 | 0.379 | 32.1, 41.7 |
| IBR-BVD-PI3-BRSV | | | | |
| T02 | 32.35 | 0-78 | | |
| IBR-BVD-PI3-BRSV- | | | | |
| MH | | | | |

The vaccinate group had a reduced percentage of clinical scores ever ≧2 at 70.3, compared with the controls at 35.1 (Table 4).

TABLE 4

| | Clinical | | Prevented | 95% |
|---|---|---|---|---|
| | Score | | Fraction | Confidence |
| Treatment | Ever ≧ 2 | Percent | (T01 vs T02) | Interval |
| T01 | 26/37 | 70.3 | 0.500 | 19.6, 71.3 |
| IBR-BVD-PI3-BRSV | | | | |
| T02 | 13/37 | 35.1 | | |
| IBR-BVD-PI3-BRSV- | | | | |
| MH | | | | |

### CONCLUSIONS

The objective of the study was to demonstrate efficacy in calves of an IBR—BVD-PI3-BRSV-MH combination against a virulent Bibersteinia trehalosi respiratory challenge. The study was valid because 70.3% ( 26/37) of the control calves tested positive for the presence of B. trehalosi in their lungs at the time of necropsy. The mortality, lung lesion, and clinical score data supports the cross-protective efficacy of the M. haemolytica fraction of the combination vaccine against a B. trehalosi challenge. Mortality was the primary efficacy variable in this study. There were 13 mortalities in the controls (35.1%), and 5 in the vaccinated animals (13.5%), with the preventive fraction estimate of 0.615 (95% CI: 5.5, 89.8), which indicates a positive effect of vaccination. Thus, the M. haemolytica fraction protected the calves against virulent B. trehalosi challenge by reducing mortality and decreasing lung lesions.

All publications cited in the specification, both patent and non-patent publications, are indicative of the level of skill of those skilled in the art to which this invention pertains. All these publications are herein fully incorporated by reference to the same extent as if each individual publication were specifically and individually indicated as being incorporated by reference.

1. A method of protecting a bovine susceptible to Bibersteinia trehalosi, the method comprising administering to said bovine an effective amount of a vaccine comprising a Mannheimia haemolytica antigen, and further comprising one or more antigens of Infectious Bovine Rhinotracheitis Virus, Bovine Viral Diarrhea Virus, Parainfluenza-3 Virus, and Bovine Respiratory Syncytial Virus.

2. The method of claim 1, wherein the vaccine comprises antigens against infectious Bovine Rhinotracheitis Virus, Bovine Viral Diarrhea Virus, Parainfluenza-3 Virus, and Bovine Respiratory Syncytial Virus.

3. The method of claim 1, wherein the Mannheimia haemolytica is killed or inactivated.

4. The method of claim 1, where the vaccine further comprises an adjuvant.

5. The method of claim 1, wherein the bovine is a bull, a steer, a calf, a cow, or a heifer.

6. The method of claim 1, wherein the Mannheimia haemolytica antigen is a toxoid or a bacterin-toxoid.

7. The method of claim 1, wherein the Mannheimia haemolytica antigen is a modified live antigen.
